(19) United States
(12) Plant Patent Application Publication
(10) Pub. No.: US 2002/0002720 P1
(43) Pub. Date: Jan. 3, 2002

(54) Gladiolus plant named 'Rosiebee Red'

(76) Inventor: John O. Zipperer III, Quito (EC)

Correspondence Address: Laura G. Barrow, Esq., P.O. Box 215, Estero, FL, 33928-0215, US

(21) Appl. No.: 09/742,726

(22) Filed: Dec. 20, 2000

(51) Int. Cl. A01H 5/00

(52) U.S. Cl. PLT/301

(57) ABSTRACT

A new and distinct gladiolus cultivar, designated ‘Rosiebee Red’, is shown and described. Compared to the ‘Advance’ variety, the ‘Rosiebee Red’ cultivar has a larger bloom size, about one to two fewer flowers per spike, and is able to maintain on the spike about four to five more flowers in open bloom.

The present invention comprises a new and distinct cultivar of a Gladiolus plant referred to by the cultivar name ‘Rosiebee Red.’

### BRIEF DESCRIPTION OF THE PHOTOGRAPHS

FIG. 1 is a photograph of two ‘Rosiebee Red’ cultivar plants in bloom.

FIG. 2 is a photograph of two ‘Rosiebee Red’ cultivar plants prior to blooming.

FIG. 3 is a photograph of a corm of the ‘Rosiebee Red’ cultivar.

Asexual Reproduction

The new cultivar was originated by the Applicant in a controlled proprietary breeding program in Ft. Myers, Fla. wherein selected gladiolus varieties were crossed. The female parent was a coral color gladiolus variety named ‘T-103’, having a length near 140 cm, a short head having about 16 florets, and a thick and heavy stem. The male parent was a red gladiolus variety named ‘Advance’, having a length of about 140 cm, floppy wide leaves, a heavy stem, and about 17-18 florets per stem. The seeds were planted in Ft. Myers, Fla., and the selection of the ‘Rosiebee Red’ cultivar was made in spring 1988. Asexual reproduction of the ‘Rosiebee Red’ cultivar was achieved by collecting cormels from the first corm. All subsequent asexual reproductions of the ‘Rosiebee Red’ are true to the original variety.

Botanical Description

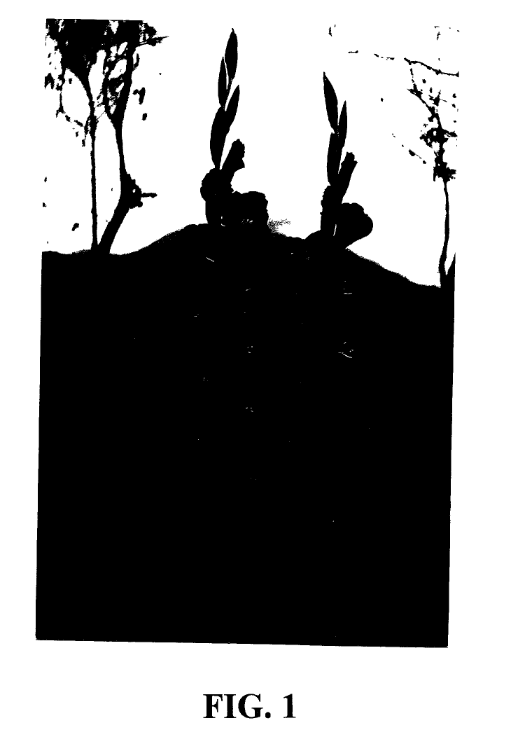

The accompanying color photograph (FIG. 1) shows the inflorescence and various stages of blooming of two ‘Rosiebee Red’ cultivar plants.

The bud size of the ‘Rosiebee Red’ cultivar, when first showing color, is 7 cm. The flowers of the ‘Rosiebee Red’ cultivar each comprise five large petals and one small petal at the bottom of the corolla. All of the petals of the corolla are present in an overlapping arrangement. Each petal has crease running up its middle. The flowers of the ‘Rosiebee Red’ cultivar are arranged on the spike of the plant on one side in a tight, overlapping zig-zag.

The corolla color of the deep portion of the flower's throat is dark red with the petals having a uniformly red color. The pistils of the flower are red, and the stamens are white. The diameter of the bloom is about 13 cm.

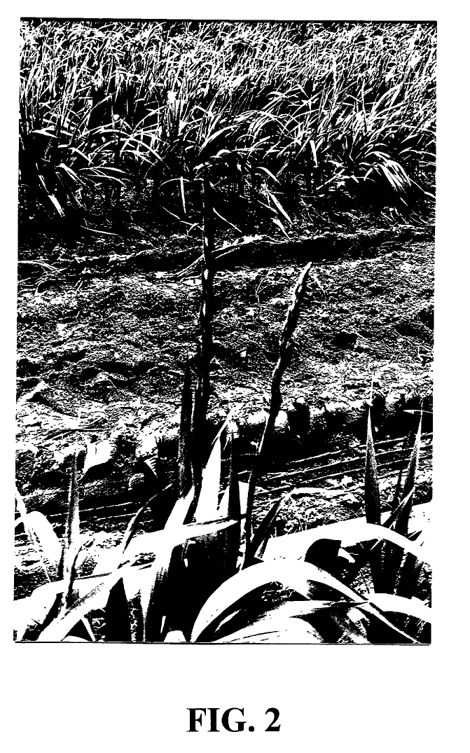

The spike has a green color tinged with red (FIG. 2). Various factors will affect spike length, however, including temperature (larger spikes occur in cooler weather), irrigation, light intensity, fertilization, soil type (larger spikes occur in heavy soils versus sandy soil), and bulb size (larger bulbs result in larger spikes). The present ‘Rosiebee Red’ cultivar is typically very long (about 140 cm) and heavy when grown in fine silt soil in Ecuador, wherein the day-lengths are twelve hours and temperatures are 75-76° F. (high) and 58-62° F. (low).

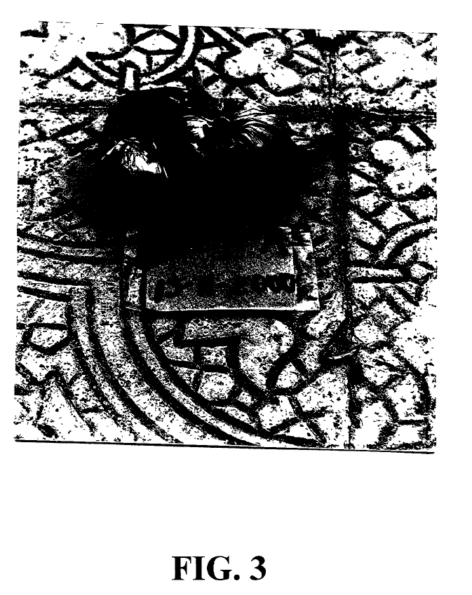

The corms of the ‘Rosiebee Red’ cultivar are typical for the gladiolus and have an orange/yellow color under the husk (FIG. 3).

The leaves of the ‘Rosiebee Red’ cultivar are wide and floppy, each tapering to a point with parallel veins running the length of the leaf. Leaf dimensions of 87 cm×4.5-5.5 cm have been observed in cultivars having jumbo bulbs with a minimum diameter of 1.75 inch.

General Observations

Compared to the gladiolus variety ‘Advance,’ the ‘Rosiebee Red’ cultivar has a larger bloom size, about one to two fewer flowers per spike, and is able to maintain on the spike about four to five more flowers in open bloom. Another distinguishing feature of the ‘Rosiebee Red’ cultivar is that can hold eight to ten flowers in open bloom, beginning with a tight cut stem.

Disease tolerance for the ‘Rosiebee Red’ cultivar is similar to the ‘Advance’ variety. Red spiders and worms are attracted to the ‘Rosiebee Red’ cultivar similar to the ‘Advance’ variety and other red gladiolus varieties. The ‘Rosiebee Red’ grows well all year long in south Florida.

The flowers of the ‘Rosiebee Red’ cultivar will bloom under long and short day lengths, having been tested down to 10.5 hours of day length in south Florida. The flowers open fast in the field; however, the cut stems may be shipped for up to a week prior to blooming. Performance under hot conditions and under winter conditions in south Florida has been good.

I claim:

1. A new and distinct gladiolus cultivar ‘Rosiebee Red’, as shown and described herein.
